(19) United States
(12) Patent Application Publication
(10) Pub. No.: US 2026/0244798 A1
(43) Pub. Date: Aug. 20, 2026

(54) METHOD AND SYSTEM FOR ESTIMATING TOOTH WEAR TYPE ON A VIRTUAL MODEL OF TEETH

(71) Applicant: 3SHAPE A/S, Copenhagen K (DK)

(72) Inventor: Jens Peter TRÄFF, Copenhagen K (DK)

(73) Assignee: 3SHAPE A/S, Copenhagen K (DK)

(21) Appl. No.: 19/164,427

(22) PCT Filed: Mar. 8, 2024
(86) PCT No.: PCT/EP2024/056243

§ 371 Date: Sep. 11, 2025

(30) Foreign Application Priority Data
Mar. 14, 2023 (DK) PA 2023 70129

(51) Int. Cl. G06F 30/12

(52) U.S. Cl. CPC G06F 30/12

(57) ABSTRACT

A computer-implemented method for detecting tooth wear on a virtual 3D model of a dental situation includes receiving, by a processor, the virtual 3D model of the dental situation, obtaining a segmented 3D model by segmenting the virtual 3D model into a plurality of individual teeth and gingiva, wherein the segmented 3D model comprises a tooth, encoding the tooth from the segmented 3D model into a latent space of a trained neural network to obtain an encoded tooth representation, wherein the latent space of the trained neural network is continuous and includes a plurality of clusters of encoded reference teeth representations, wherein each cluster from the plurality of clusters of encoded reference teeth representations is representative of a tooth wear type.

### TECHNICAL FIELD

The disclosure relates to a computer-implemented method and system for estimating tooth wear type on a virtual 3D model of a dental situation. A trained neural network is used to estimate tooth wear type present on a tooth from the dental situation.

### BACKGROUND

Tooth wear is a dental condition characterizing loss of tooth structure. It is often painful and impairs the function of teeth. Three types of tooth wear occurring are abrasion, attrition, and erosion. Abrasion is physical wear of teeth caused by a factor other than tooth-to-tooth contact, such as inappropriate toothbrushing. Attrition is loss of tooth structure from tooth-to-tooth contact. Dental erosion, referred to also as erosion, is dissolving of tooth enamel due to the presence of acids in the mouth.

Damage caused by tooth wear is irreversible and can be difficult to repair. Timely detection and monitoring of tooth wear by general practitioners are therefore essential for preserving tooth structure.

Clinical detection and diagnosis of tooth wear is currently based on direct visual inspections of teeth, which are very subjective. Visual inspections are characterized by low sensitivity and low reproducibility when performed by general practitioners. Furthermore, tooth substance loss is visually detectable only when a significant amount of hard dental tissue is already lost.

Digital dentistry and use of intraoral scanners enabled more accurate dental condition detection methods to be developed, thereby reducing, or completely removing, practitioner's subjectivity. For example, it is possible today to acquire two intraoral scans of a patient's dental situation at different time periods. It is then possible to detect tooth wear by comparing geometries of the two scans and identifying quantifiable differences. The drawback of this method is that tooth wear cannot be determined from only a single scan.

There is a clear need to develop methods and systems which will aid in tooth wear detection on a single scan of intraoral situation of the patient and further enable reliable classification of tooth wear into established tooth wear types.

### SUMMARY

Disclosed herein is a computer-implemented method for detecting tooth wear on a virtual 3D model of a dental situation, the method comprising:
receiving, by a processor, the virtual 3D model of the dental situation,
obtaining a segmented 3D model by segmenting the virtual 3D model into a plurality of individual teeth and gingiva, wherein the segmented 3D model comprises a tooth,
encoding the tooth from the segmented 3D model into a latent space of a trained neural network to obtain an encoded tooth representation, wherein the latent space of the trained neural network is continuous and comprises a plurality of clusters of encoded reference teeth representations, wherein each cluster from the plurality of clusters of encoded reference teeth representations is representative of a tooth wear type,
comparing the encoded tooth representation to each cluster from the plurality of clusters of encoded reference teeth representations to determine a cluster comprising the encoded tooth representation,
assigning the tooth wear type to the tooth based on the determined cluster,
displaying the tooth with the assigned tooth wear type.

In an embodiment of the disclosure a computer-implemented method for detecting tooth wear on the virtual 3D model of the dental situation is disclosed, the method comprising:
receiving, by the processor, the virtual 3D model of the dental situation,
obtaining the segmented 3D model by segmenting the virtual 3D model into the plurality of individual teeth and gingiva, wherein the segmented 3D model comprises the tooth,
encoding the tooth from the segmented 3D model into the latent space of the trained neural network to obtain the encoded tooth representation,
encoding a plurality of reference teeth cases into the latent space of the trained neural network to obtain encoded reference teeth representations, wherein each reference tooth case from the plurality of reference teeth cases comprises a reference tooth and optionally a tooth wear type label corresponding to the reference tooth,
clustering the encoded reference teeth representations according to their corresponding tooth wear type labels to obtain the plurality of clusters of encoded reference teeth representations such that each cluster from the plurality of clusters of encoded reference teeth representations is representative of a tooth wear type,
comparing the encoded tooth representation to each cluster from the plurality of clusters of encoded reference teeth representations to determine a cluster comprising the encoded tooth representation,
assigning the tooth wear type to the tooth based on the determined cluster,
displaying the tooth with the assigned tooth wear type, wherein the latent space of the trained neural network is continuous.

Expression “3D” throughout the present disclosure refers to a term “three-dimensional”. Similarly, term “2D” refers to a term “two-dimensional”. Term “virtual 3D model of a dental situation” refers to a virtual, three-dimensional, computer-generated representation of the patient's dental situation. The virtual 3D model may also be referred to as a digital 3D model.

Such virtual 3D model may be constructed, by the processor, based on scan data collected in an intraoral scanning process in which an intraoral scanner may be used to scan the patient's dental situation comprising teeth and gingiva. The virtual 3D model can alternatively be generated by using a conventional 3D scanner, a so-called lab-or desktop scanner, to scan a gypsum model of the patient's dental situation. The virtual 3D model can be stored in a memory of a computer system, for example in a Standard Triangle Language (STL) format.

According to the method of the disclosure the virtual 3D model is received by the processor. The process of performing 3D scanning is not necessarily a part of the method. The 3D scanning may be performed by a dental professional using the intraoral scanner, or the conventional 3D scanner.

The virtual 3D model can be received or accessed by the processor. The virtual 3D model may usually be displayed on a display screen in a form of a 3D mesh, a point cloud, a graph, a volumetric representation, or any other suitable 3D representation form.

Further, the method may comprise obtaining a segmented 3D model by segmenting the virtual 3D model into a plurality of individual teeth and gingiva. The segmented 3D model may comprise at least a tooth. The tooth may in this way be isolated from rest of the teeth and gingiva, and may subsequently be analyzed for tooth wear presence by utilizing methods according to the disclosure.

Segmenting the virtual 3D model may be performed via a segmentation process which allows for identification of distinct dental objects such as individual teeth and/or surrounding gingiva in the virtual 3D model. Individual teeth can be assigned a tooth identifier, for example according to the Universal Numbering Notation (UNN) in which numerals 1-32 may be assigned to human teeth. The segmentation process may comprise use of algorithms such as Principal Component Analysis (PCA) or harmonic fields. The segmentation process may alternatively or additionally comprise use of machine learning models.

Further, the method may comprise encoding the tooth from the segmented 3D model into the latent space of the trained neural network to obtain the encoded tooth representation, wherein the latent space of the trained neural network may be continuous and may comprise the plurality of clusters of encoded reference teeth representations, wherein each cluster from the plurality of clusters of encoded reference teeth representations is representative of the tooth wear type. The plurality of clusters of encoded reference teeth representations may thus serve as reference information which may be used to determine the tooth wear type present on the tooth from the segmented 3D model. The encoded tooth representation may thus be mapped against this reference information which may have been previously encoded into the trained neural network.

The trained neural network may be a variational autoencoder network comprised of an encoder and a decoder. Variational autoencoders are regularized versions of autoencoders and may allow for new content generation based on input provided. The encoder and/or the decoder may be convolutional neural networks.

Encoding the tooth may refer to applying the trained neural network to the tooth from the segmented 3D model. The tooth may be input into the encoder of the trained neural network to obtain the encoded tooth representation. The encoded tooth representation may be a set of latent space variables, for example a set of scalar numbers, representing the tooth in the latent space of the trained neural network. Latent space variables may also be referred to as latent space parameters.

Encoding the tooth from the segmented 3D model into the latent space of the trained neural network may comprise feeding the information about geometry of the tooth into the trained neural network.

The tooth from the segmented 3D model may be in 3D format, such as a point cloud, a graph, a volume or a 3D mesh. The tooth in the 3D mesh format may be referred to as a 3D tooth mesh.

The trained neural network may, in an embodiment, be suitable for processing input in 2D format. For that purpose, the tooth may be transformed from 3D format into 2D format. One example of 2D format of the tooth may be a flattened planar mesh, obtained by transforming the 3D tooth mesh into a 2D tooth mesh. This transformation process may be referred to as “mesh flattening” and may transform the tooth from its 3D format into 2D format without loss of information on three-dimensional placement of vertices, edges and faces of the 3D tooth mesh.

The tooth from the segmented 3D model may thus be represented by the latent space variables in the latent space of the trained neural network. The latent space variables may be scalar numbers.

In general, a latent space of a neural network may be understood as an embedding space comprising latent space variables which represent encoded input. The latent space variables resembling each other more closely are positioned closer to one another in the latent space relative to more differing latent space variables.

The latent space of the trained neural network may be continuous. Continuity of the latent space is a property of the trained neural network that characterizes a relationship of data points in the latent space representing input data, on one side, and outputs obtained after decoding the data points with the decoder, on the other side. For example, two close points in the continuous latent space result in closely related content once decoded.

The latent space of the trained neural network may comprise a plurality of clusters of encoded reference teeth representations, wherein each cluster from the plurality of clusters of encoded reference teeth representations may be representative of a tooth wear type.

The encoded reference teeth representations may also be referred to as initial latent space variables and may be obtained by encoding reference information into the trained neural network, as will be explained later. The reference information may be a plurality of reference teeth cases, where each reference tooth case from the plurality of reference teeth cases may comprise a reference tooth in 2D or a 3D representation. Optionally, each reference tooth case may comprise a tooth wear type label assigned to the corresponding reference tooth. The tooth wear type label may be assigned manually to the corresponding reference tooth, for example by a dental practitioner. For example, the tooth wear type label assigned to the reference tooth may be one or more of following: “erosion”, “abrasion”, “attrition”, “no tooth wear”. Additionally, severity levels such as “mild”, “moderate”, “severe” may be used in the tooth wear type label. Throughout the disclosure the tooth wear type label corresponding to the reference tooth may also be referred to as a reference tooth wear type label.

Number of the reference teeth cases may be at least one thousand, preferably at least ten thousand, more preferably at least fifty thousand. The number of reference teeth cases used for obtaining the encoded reference teeth representations may directly impact efficiency of the trained neural network in assigning correct tooth wear type to the provided input. The encoded reference teeth representations may serve as basis for correctly classifying tooth wear type of new input data to which the trained neural network may be applied to.

The plurality of clusters of these obtained encoded reference teeth representations may be obtained by performing a statistical analysis on the encoded reference teeth representations. The performed statistical analysis may result in identifying dependence of the encoded reference teeth representations to the tooth wear type labels corresponding to the encoded reference teeth representations.

Moreover, since the latent space of the trained neural network may be continuous, a path between the obtained clusters may be defined. This path may be piece-wise linear between representative data points of the obtained clusters and may be mathematically described. This obtained path may allow changing the tooth wear type label of a specific tooth representation, for example changing the tooth wear type label from a value “abrasion” to the value “no tooth wear”. It may then be possible to reconstruct an ideal model of that specific tooth representation, where no tooth wear is present.

In an embodiment of the disclosure, each reference tooth case of the plurality of reference teeth cases may be transformed, using the trained neural network, through the different types of tooth wear such as “erosion”, “abrasion”, “attrition”, “no tooth wear”. Additionally, each reference tooth case of the plurality of reference teeth cases may further be transformed through different tooth wear severities such as “mild”, “moderate”, “severe” or “no tooth wear”. Thereby, a plurality of reference teeth representations may be obtained from each reference tooth case. As an effect, a piece-wise linear trajectory of different tooth wear types and/or severity levels for each reference tooth case may be obtained. Further, all reference tooth cases of the same type (e.g. molars) may be grouped and an average of the corresponding trajectories can be determined. This average of the corresponding trajectories may serve as a vector that can be used to change the different tooth wear type of a tooth.

Clustering of the encoded reference teeth representations may be performed according to a common trait of the tooth cases, which may be the tooth wear type label associated with reference teeth. As mentioned earlier, the tooth wear type may be abrasion, attrition and/or erosion. The tooth wear type in the context of the disclosure may, additionally or alternatively, be absence of tooth wear. The effect of the clustering may be that the encoded reference teeth representations are grouped according to the tooth wear type label associated with the reference teeth represented by the encoded reference teeth representations.

Therefore, the method according to an embodiment may comprise obtaining a cluster of at least a first part of the encoded reference teeth representations corresponding to abrasion. The method may further comprise obtaining a cluster of at least a second part of the encoded reference teeth representations corresponding to attrition. Further, the method may comprise obtaining a cluster of at least a third part of the encoded reference teeth representations corresponding to erosion. The method may additionally comprise obtaining a cluster of at least a fourth part of the encoded reference teeth representations corresponding to tooth wear absence. As a result, the encoded reference teeth representations may be clustered according to the tooth wear type label of the reference teeth cases. In an example, the latent space of the trained neural network may comprise the encoded reference teeth representations grouped in four clusters.

The method according to an embodiment may further comprise comparing the encoded tooth representation to each cluster from the plurality of clusters of encoded reference teeth representations to determine a cluster of encoded reference teeth representations comprising the encoded tooth representation. In this way, the encoded tooth representation is classified into a nearest cluster of encoded reference teeth representations.

By determining the nearest cluster, the tooth wear type corresponding to the tooth from the segmented 3D model may be estimated. For example, a result of the comparing step may be that the encoded tooth representation lies in a cluster of encoded reference teeth representations corresponding to attrition. Subsequently to the comparing step, the corresponding tooth wear type may be assigned to the tooth from the segmented 3D model. For example, attrition may be assigned to the tooth from the segmented 3D model.

In an embodiment, comparing the encoded tooth representation to each cluster from the plurality of clusters of encoded reference teeth representations may comprise using a nearest neighbor search algorithm.

The method may further comprise assigning the tooth wear type to the tooth from the segmented 3D model based on the determined nearest cluster. For example, tooth wear type value “attrition” may be assigned to the tooth from the segmented 3D model if the latent space variables representing the tooth belong to the cluster of encoded reference teeth representations corresponding to attrition.

The method according to an embodiment may further comprise displaying the tooth from the segmented 3D model with the assigned tooth wear type. Displaying may occur in form of a text alert indicating the detected tooth wear type. Alternatively or additionally, displaying may occur by coloring the tooth in the segmented 3D model in a color different to color of rest of the teeth of the segmented 3D model.

By displaying the tooth, the dental practitioner may easily focus on inspecting the tooth in question and/or decide on a further treatment option. Alternatively or additionally, the further treatment option may be provided automatically, depending on the detected tooth wear type. For example, if the detected tooth wear type is abrasion, the message may be generated indicating that the patient should change their toothbrush to a soft toothbrush to reduce rate of abrasion.

Therefore, the method according to an embodiment may comprise applying the trained neural network to the tooth from the segmented 3D model of the dental situation, estimating the tooth wear type of the tooth and assigning the tooth wear type to the tooth. Alternatively or additionally, the further treatment option may be generated.

Thus, the method according to the disclosure may allow for determination of the tooth wear type associated with a tooth that is input into the trained neural network, by using clustered reference information in the latent space of the trained neural network.

An advantage of detecting tooth wear type on the tooth via the method of the disclosure is reflected in that various types of tooth wear can be precisely and reliably detected. In contrast, manual inspection for tooth wear by the dental practitioner may not be objective, as certain types of tooth wear and/or various severity levels of tooth wear may be overlooked, unless the tooth wear is severe. Even when the tooth wear present on the tooth is severe, the method according to the disclosure provides clear classification of tooth wear type and assessment of the health status that can be easily presented to the patient. For example, it may be communicated to the patient that a certain tooth suffers both from mild abrasion and mild attrition.

According to an embodiment, encoding the tooth from the segmented 3D model may comprise encoding a sampled matrix representing the tooth from the segmented 3D model.

The sampled matrix may be obtained by sampling the planar tooth mesh, wherein the planar tooth mesh may be a 2D representation of the tooth from the segmented 3D model. For example, a raw three-dimensional scan of the surface of the tooth may have tens of thousands of vertices. By sampling the planar tooth mesh, the number of vertices may be significantly reduced, while still preserving the information of the three-dimensional object scanned.

The planar tooth mesh may be obtained by flattening the 3D tooth mesh, wherein the 3D tooth mesh may represent the tooth from the segmented 3D model.

The encoded tooth representation may also be referred to as the set of latent space variables representing the tooth. The set of latent space variables may therefore be a representation of the tooth in the latent space of the trained neural network. This set of latent space variables may be a set of scalar numbers.

In an embodiment, the trained neural network may be a variational autoencoder. In another embodiment, the trained neural network may be a normalizing flow.

Each tooth case may comprise the reference tooth, in 2D or 3D format, and the tooth wear type label associated to the reference tooth. By encoding the plurality of reference teeth cases, the encoded reference teeth representations may be obtained in the latent space of the trained neural network.

Method according to an embodiment may comprise obtaining the plurality of clusters of the encoded reference teeth representations by performing the statistical analysis on the encoded reference teeth representations. The statistical analysis may be a clustering analysis.

The statistical analysis on the encoded reference teeth representations may be performed to determine a correlation between encoded reference teeth representations and the tooth wear type labels.

In an embodiment, performing the statistical analysis on the encoded reference teeth representations may comprise using a Principal Component Analysis algorithm.

In another embodiment, performing the statistical analysis on the encoded reference teeth representations may comprise performing a tabulation analysis. Other clustering algorithms may also be employed such as machine learning clustering techniques, for example.

As a result of performing the statistical analysis on the encoded reference teeth representations in the latent space, clustering of the encoded reference teeth representations may be obtained, such that the encoded reference teeth representations representing reference teeth suffering of same tooth wear type may be clustered closer together relative to the encoded reference teeth representations representing reference teeth suffering of a different tooth wear type. For example, the encoded reference teeth representations characterized by “abrasion” tooth wear type may be clustered in one cluster different from another cluster characterized by “attrition” tooth wear type.

A decision boundary may be created separating different clusters of the encoded reference teeth representations. If a new tooth is encoded into the trained neural network, a new set of latent space variables may be obtained. The decision boundary may then allow for determining which cluster of the encoded reference teeth representations the new set latent space variables belongs to. In this way, the tooth wear type of the new tooth may be estimated.

A statistical parameter, such as a mean and/or a median may be determined for each cluster of the plurality of clusters of encoded reference teeth representations as a representative of the tooth wear type within the each cluster. This statistical parameter may be used as a target for performing the proximity search to determine where the new set of latent space variables, representing the new encoded tooth case, belongs in terms of already determined clusters of the encoded reference teeth representations. The statistical parameter may serve as a direct target for the proximity search or may be used in determining a decision boundary in the proximity search.

In an embodiment of the disclosure, disclosed is a data processing apparatus comprising means for carrying out a method according to any described embodiment.

The data processing apparatus may comprise means for carrying out the method comprising:
receiving, by the processor, the virtual 3D model of the dental situation,
obtaining the segmented 3D model by segmenting the virtual 3D model into the plurality of individual teeth and gingiva,
encoding the tooth from the segmented 3D model into the latent space of the trained neural network to obtain the encoded tooth representation, wherein the latent space of the trained neural network is continuous and comprises the plurality of clusters of encoded reference teeth representations, wherein each cluster from the plurality of clusters of encoded reference teeth representations is representative of the tooth wear type,
comparing the encoded tooth representation to each cluster from the plurality of clusters of encoded reference teeth representations to determine the cluster comprising the encoded tooth representation,
assigning the tooth wear type to the tooth from the segmented 3D model based on the determined cluster,
displaying the tooth from the segmented 3D model with the assigned tooth wear type.

In a further embodiment of the disclosure, disclosed is a computer program product comprising instructions which, when the program is executed by a computer, causes the computer to carry out the method of any described embodiment.

The computer program product may comprise instructions which, when the program is executed by a computer, causes the computer to carry out the computer-implemented method comprising:
receiving, by the processor, the virtual 3D model of the dental situation,
obtaining the segmented 3D model by segmenting the virtual 3D model into the plurality of individual teeth and gingiva,
encoding the tooth from the segmented 3D model into the latent space of the trained neural network to obtain the encoded tooth representation, wherein the latent space of the trained neural network is continuous and comprises the plurality of clusters of encoded reference teeth representations, wherein each cluster from the plurality of clusters of encoded reference teeth representations is representative of the tooth wear type,
comparing the encoded tooth representation to each cluster from the plurality of clusters of encoded reference teeth representations to determine the cluster comprising the encoded tooth representation,
assigning the tooth wear type to the tooth from the segmented 3D model based on the determined cluster,
displaying the tooth from the segmented 3D model with the assigned tooth wear type.

In yet a further embodiment of the disclosure, disclosed is a non-transitory computer readable medium comprising instructions which, when executed by a computer, cause the computer to carry out the method according to any of described embodiments.

The non-transitory computer readable medium may comprise instructions which, when executed by a computer, cause the computer to carry out the computer-implemented method comprising:
receiving, by the processor, the virtual 3D model of the dental situation,
obtaining the segmented 3D model by segmenting the virtual 3D model into the plurality of individual teeth and gingiva,
encoding the tooth from the segmented 3D model into the latent space of the trained neural network to obtain the encoded tooth representation, wherein the latent space of the trained neural network is continuous and comprises the plurality of clusters of encoded reference teeth representations, wherein each cluster from the plurality of clusters of encoded reference teeth representations is representative of the tooth wear type,
comparing the encoded tooth representation to each cluster from the plurality of clusters of encoded reference teeth representations to determine the cluster comprising the encoded tooth representation,
assigning the tooth wear type to the tooth from the segmented 3D model based on the determined cluster,
displaying the tooth from the segmented 3D model with the assigned tooth wear type.

In the following section, a training process for the trained neural network is described. In further disclosure, term “neural network” is used to refer to non-trained neural network i.e. the trained neural network prior to completing the training process. Phrase “completing the training process” may be understood as determining weights of the trained neural network such that the trained neural network is suitable for application in embodiments described throughout the disclosure.

The training process may start by receiving training data. The training data may comprise a plurality of training teeth cases, wherein each training tooth case from the plurality of training teeth cases may comprise a training tooth, for example in 3D format or 2D format and optionally an associated known training age parameter. A training age parameter may be understood as an age parameter corresponding to the training tooth. The plurality of training teeth cases may be comprised of more than one hundred thousand training teeth cases, or more preferably more than five hundred thousand training teeth cases.

The training data may comprise a set of labels describing tooth wear type associated to each of the plurality of training teeth cases and/or a set of labels describing tooth wear severity associated to each of the plurality of training teeth cases. The set of labels may be manually assigned to the plurality of training teeth cases, for example by dental practitioners. In general, all different types of tooth wear may be represented in the training data.

The plurality of training teeth cases may be obtained by scanning jaws of plurality of patients. Thereby, a plurality of jaw scans may be obtained, which may further be segmented into the plurality of training teeth. In the training data, the training age parameters corresponding to the training teeth may be known. For example, a training tooth associated with a patient who is twenty years old has a training age parameter with a value of twenty years.

The training data may comprise the training teeth cases having training age parameters in a range from 10 years of age to 90 years of age. By having a wide range of training teeth ages, the training process of the neural network may be improved as well as the overall efficiency of the neural network in its subsequent application. In general, all age groups that may be relevant to cover by the subsequent application of the neural network may be represented in the training data.

If the training teeth are in 3D format such as a 3D mesh, then the training teeth may be first converted in 2D format to obtain a 2D representation of each of the training teeth. This conversion may be referred to as “mesh flattening” and is described throughout the disclosure. Alternatively, the neural network may be suitable for processing directly data in 3D format.

Through this pre-processing stage of the training data, sampled matrices representing the training teeth may be obtained which may then be fed into the neural network as an input. This may be referred to as encoding the training data via an encoder of the neural network to obtain latent space variables corresponding to the training data.

An output of the neural network may be obtained by decoding the obtained latent space variables corresponding to the training data, via a decoder of the neural network. The output may be compared to the input to obtain a reconstruction loss describing a difference between the input and the output.

Additionally, a distribution of the latent space variables corresponding to the training data may be obtained and compared to a desired prior. The desired prior may, in an embodiment, be a standard normal distribution. This comparison may provide a statistical distance loss measuring a difference between two distributions.

The reconstruction loss and the statistical distance loss may be used together as a combined loss to train the neural network.

In an embodiment, a regularization term, known as Kullback-Leibler divergence or KL loss, may be used as the statistical distance loss that can measure the difference between two distributions.

The reconstruction loss may comprise a combined structured similarity index measure (SSIM) and L1 loss function used to minimize the error which is the sum of all absolute differences between a true value and a predicted value. SSIM measure and L1 loss function may be weighted adjustably, for example with 84% allocation towards the SSIM measure. This value may provide for suitable prioritization of a structure of the output provided by SSIM measure, over an absolute value of pixels in the output provided by L1 loss function.

The training process may be reiterated until convergence is achieved on the combined loss after which the neural network becomes trained.

The training data may be curated by filtering out artifacts such as fillings, inlays, onlays and/or braces. This curating of the training data focuses the neural network on the effect of light to moderate tooth wear.

Validation data may be used to validate performance of the trained neural network. The validation data may be different to the training data.

During the training process, multiple hyperparameter configurations may be used, one example being an “ADAM” optimizer with a learning rate of 1e−4 and using a learning rate scheduler that decays learning rates on plateaus.

### BRIEF DESCRIPTION OF THE FIGURES

Aspects of the disclosure may be best understood from the following detailed description taken in conjunction with the accompanying figures. The figures are schematic and simplified for clarity, showing certain details to improve the understanding of the claims, while other details are left out. The individual features of each embodiment may each be combined with any or all features of other embodiments. These and other embodiments, features and/or technical effects will be apparent from and elucidated with a reference to the illustrations described hereinafter in which:

FIG. 1 illustrates a virtual 3D model of a dental situation.

FIG. 2 illustrates a flow chart of a method according to an embodiment.

FIG. 3A-3B illustrate the trained neural network according to an embodiment where the trained neural network comprises an encoder and a decoder, as well as a possible input to, and output from the trained neural network.

FIG. 4 illustrates steps of transforming a 3D tooth mesh into 2D format for use as the input to the trained neural network.

FIG. 5A-5B illustrate n-dimensional latent space of the trained neural network, visualized as a 2D space. Different clusters of encoded reference teeth representations are shown, as well as piece-wise linear path between the clusters.

FIG. 6 illustrates a dental scanning system according to an embodiment.

FIG. 7 illustrates an exemplary workflow of the patient's visit to the dental practitioner.

### DETAILED DESCRIPTION

In the following description, reference is made to the accompanying figures, which show by way of illustration how the invention may be practiced.

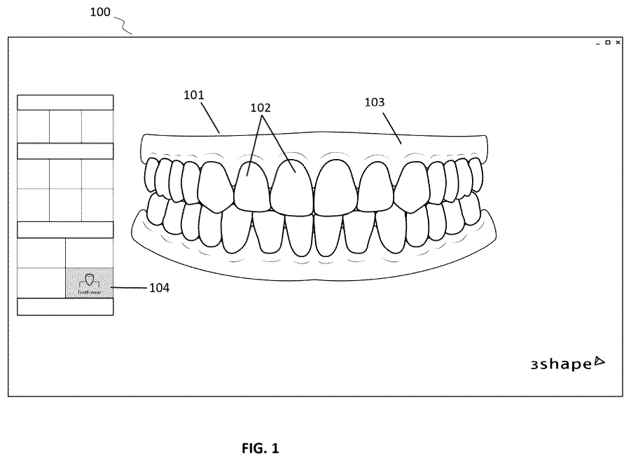

FIG. 1 illustrates a user interface 100 with a displayed virtual 3D model 101 of a dental situation of a patient. The virtual 3D model 101 may be displayed on a display screen in a form of a 3D mesh, a point cloud, a 3D graph, a volumetric representation, or any other suitable 3D representation form. The virtual 3D model 101 may be representative of the dental situation of the patient, i.e. the virtual 3D model 101 may comprise combined representations of teeth and gingiva of the patient's dental situation.

The user interface 100 may comprise a button 104 which, once engaged by a user, initiates the method for estimation of tooth wear type present on teeth of the virtual 3D model 101. The method may, alternatively or additionally, be initiated automatically, without user engagement.

To identify and separate individual teeth 102 and gingiva 103 within the virtual 3D model 101, the virtual 3D model 101 may be segmented. Segmentation may refer to identifying facets of the 3D mesh representation of the virtual 3D model 101 belonging to individual teeth 102 as per Universal Numbering System/Notation (UNN). In this way individual teeth 102, or parts of the teeth 102, may be analyzed for presence of different tooth wear types.

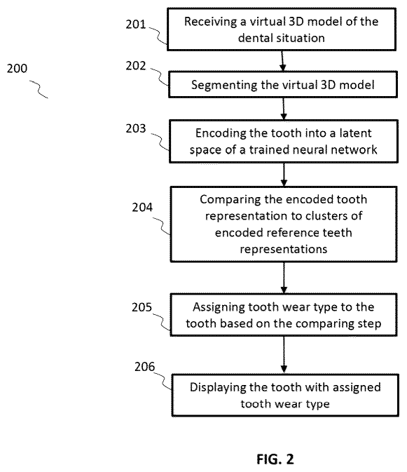

FIG. 2 shows a flowchart illustrating a method 200 according to an embodiment.

First, the virtual 3D model 101 may be received, by the processor, in step 201. The virtual 3D model 101 can be stored in a memory of a computer system, for example in a Standard Triangle Language (STL) format. The virtual 3D model 101 can be received by the processor, for example when the user engages the button 104 in the user interface 100. The virtual 3D model 101 may usually be displayed on the display screen in a form of the 3D mesh, the point cloud, the 3D graph, the volumetric representation, or any other suitable 3D representation form.

Step 202 illustrates segmenting the virtual 3D model 101 to obtain a segmented virtual 3D model. The segmented virtual 3D model comprises at least one tooth 102 from a plurality of individual teeth belonging to the virtual 3D model 101. The segmented virtual 3D model may comprise additional teeth and/or gingiva 103. Segmentation of the virtual 3D model 101 may be achieved in several ways. According to an example, segmenting may comprise use of surface curvatures to identify boundaries of tooth representations. A curvature threshold value can be selected to distinguish tooth boundary regions from the rest of surface of the virtual 3D model 101.

In another example, segmentation of the virtual 3D model 101 may comprise use of a harmonic field to identify tooth boundaries. On the virtual 3D model 101, a harmonic field may be a scalar attached to each mesh vertex satisfying the condition: Δφ=0, where Δ is Laplacian operator, subject to Dirichlet boundary constraint conditions. Above equation may be solved, for example using least squares method, to calculate the harmonic field.

Segmented teeth 102 can then be extracted by selecting optimal isolines connecting datapoints with same value as tooth representation boundaries.

In yet another example, segmenting the virtual 3D model 101 may comprise use of a segmentation machine learning model. In particular, the virtual 3D model 101 may be converted into a series of 2D virtual images. The segmentation machine learning model may be applied to the series of 2D virtual images. For each 2D virtual image, segmentation can be performed to distinguish between different teeth classes and gingiva. After classification of each element of each 2D virtual image, back-projection onto the virtual 3D model 101 may be performed. This method for segmenting the virtual 3D model 101 may be advantageous as the segmentation machine learning model utilizes the series of 2D virtual images, overall resulting in fast and accurate facet classification.

Step 203 of the method 200 illustrates encoding the tooth 102 from the segmented virtual 3D model into a latent space 302 of a trained neural network 300. In this way, an encoded tooth representation may be obtained in form of latent space variables. Encoding may refer to applying the trained neural network 300, or more specifically an encoder 301 of the trained neural network 300, to the tooth 102 from the segmented 3D model. The encoded tooth representation may be referred to as a set of latent space variables representing the tooth 102 in the latent space 302 of the trained neural network 300.

The latent space 302 of the trained neural network 300 may be well understood as an embedding space comprising latent space variables representing encoded input data, wherein the latent space variables resembling each other more closely are positioned closer to one another in the latent space 302.

The latent space 302 of the trained neural network 300 may be continuous. Continuity of the latent space 302 is a property that characterizes a relationship of data points in the latent space 302 and an output 307 which may be obtained after decoding those data points with the decoder 303. For example, two close data points in the continuous latent space 302 result in closely related content once decoded. Continuity of the latent space 302 may enable clustering analysis of the latent space variables within the latent space 302.

The latent space 302 of the trained neural network 300 may comprise a plurality of clusters 503-506 of encoded reference teeth representations, wherein each cluster from the plurality of clusters 503-506 of encoded reference teeth representations may be representative of a tooth wear type.

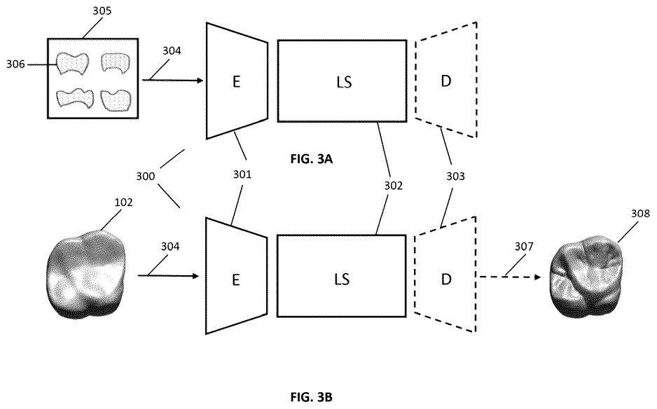

This plurality of clusters of encoded reference teeth representations may be obtained by encoding reference information into the trained neural network 300, as shown in FIG. 3A. The reference information may be a plurality of reference teeth cases 305, where each reference tooth case 306 from the plurality of reference teeth cases 305 may comprise a 2D or a 3D representation of a reference tooth. Each reference tooth case 306 may further comprise information on tooth wear type associated with the reference tooth of that specific tooth case 306. This information on tooth wear type may be a tooth wear type label which may be assigned manually to each reference tooth, for example by the dental practitioner.

Number of reference teeth cases in the plurality of reference teeth cases 305 may be at least one thousand, preferably at least ten thousand, more preferably at least fifty thousand. The number of reference teeth cases may directly impact efficiency of the trained neural network 300 in estimating correct tooth wear type for a newly provided input.

The clusters 503-506 of these obtained encoded reference teeth representations may be obtained by performing clustering analysis on the encoded reference teeth representations.

The clustering may be performed according to a value of the tooth wear type label associated with reference teeth in the plurality of reference teeth cases 305. As mentioned earlier, the value of the tooth wear type label may be one or more of “abrasion”, “attrition” “erosion” and/or “no tooth wear”. The tooth wear type in the context of the disclosure may be, additionally or alternatively, absence of tooth wear. The effect of the clustering may be that the encoded reference teeth representations become grouped according to the corresponding value of tooth wear type label.

The method, according to an embodiment, may thus comprise clustering the encoded reference teeth representations according to the tooth wear type labels corresponding to the plurality of reference teeth cases 305.

Reference teeth within the plurality of reference teeth cases 305 may be manually labeled for presence of tooth wear type.

The method according to an embodiment may comprise obtaining a cluster 503 of at least a first part of the encoded reference teeth representations corresponding to abrasion. The method may further comprise obtaining a cluster 504 of at least a second part of the encoded reference teeth representations corresponding to attrition. Further, the method may comprise obtaining a cluster 505 of at least a third part of the encoded reference teeth representations corresponding to erosion. The method may additionally comprise obtaining a cluster 506 of at least a fourth part of the encoded reference teeth representations corresponding to tooth wear absence. As a result, the encoded reference teeth representations representing the plurality of reference teeth cases 305 may be clustered according to the tooth wear type label value corresponding to the plurality of reference teeth cases 305.

The method 200, according to an embodiment, may further comprise step of comparing 204 the encoded tooth representation to each cluster from the plurality of clusters of encoded reference teeth representations 503-506 to determine a cluster of the reference teeth representations comprising the encoded tooth representation.

By determining the cluster of the reference teeth representations comprising the encoded tooth representation, the tooth wear type corresponding to the tooth 102 from the segmented 3D model may be estimated. For example, a result of the comparing step may be that the encoded tooth representation is associated to a cluster 504 corresponding to attrition. Subsequently to the comparing step, the estimated tooth wear type may be assigned to the tooth 102 from the segmented 3D model. For example, attrition may be assigned to the tooth 102 from the segmented 3D model.

In an embodiment, comparing the encoded tooth representation to at least one cluster from the plurality of clusters 503-506 of the reference teeth representations in the latent space 302 of the trained neural network 300 may comprise using nearest neighbor search algorithm.

The method 200 may further comprise step of assigning 205 the tooth wear type to the tooth 102 from the segmented 3D model based on the determined cluster. For example, attrition may be assigned to the tooth 102 from the segmented 3D model because the latent space variables representing the tooth 102 belong to the cluster 504 corresponding to attrition.

The method 200 according to an embodiment may further comprise step of displaying 206 the tooth 102 from the segmented 3D model with the assigned tooth wear type. Displaying 206 may occur in form of a text alert indicating the detected tooth wear type. Alternatively or additionally, displaying may occur by coloring the tooth 102 in the segmented 3D model in a color different to rest of the teeth of the segmented 3D model.

In this way, the dental practitioner may easily focus on the tooth 102 in question, inspect the tooth 102 further and/or decide on a further treatment option. Alternatively or additionally, the further treatment option may be provided automatically, depending on the detected tooth wear type. For example, if the detected tooth wear type is abrasion, the message may be generated indicating that the patient should change their toothbrush to a soft toothbrush to reduce rate of abrasion.

FIGS. 3A and 3B both illustrate components of the trained neural network 300 used in an embodiment according to the disclosure. The trained neural network 300 may comprise an encoder 301 and optionally a decoder 303. Additionally, the latent space 302 is illustrated in the figures. The trained neural network 300 may be the same in both FIG. 3A and FIG. 3B. FIG. 3A illustrates encoding process of the plurality of reference teeth cases 305. The plurality of reference teeth cases 305 serves as the input 304 to the trained neural network 300. The decoder 303 may be inactive, thus illustrated with a dashed line.

Once encoding of the plurality of reference teeth cases 305 is performed and the plurality of clusters 503-506 has been determined, the trained neural network 300 may be ready for application on a new set of input data. This is illustrated in FIG. 3B. Here, the tooth 102 from the segmented virtual 3D model may serve as the input 304 to the trained neural network 300 for the purpose of estimating tooth wear type of the tooth 102. In FIG. 3B the decoder 303 may be inactive, thus illustrated with a dashed line.

Encoder 301 of the trained neural network 300 may be a convolutional neural network or a dense neural network. The function of the encoder 301 is to convert the input 304 into a set of latent variables. The input 304 may be the tooth 102 from the segmented virtual 3D model. By encoding the input 304, the encoded tooth representation may be obtained, comprising latent space variables representing the tooth 102 in the latent space 302 of the trained neural network 300.

The latent space variables may act as a parametrization of the tooth 102, both for embodying the tooth 102 and for transforming the tooth 102. The latent space variables may be scalar numbers. Thereby, the latent space variables may allow the shape of the tooth 102 to be represented by a set of scalar numbers. These numbers may not be easily interpreted by human perception but do contain information about the shape of the tooth 102 as discovered by the machine learning method. Further, the set of scalar numbers may be translated back into a corresponding 3D representation of the tooth 102.

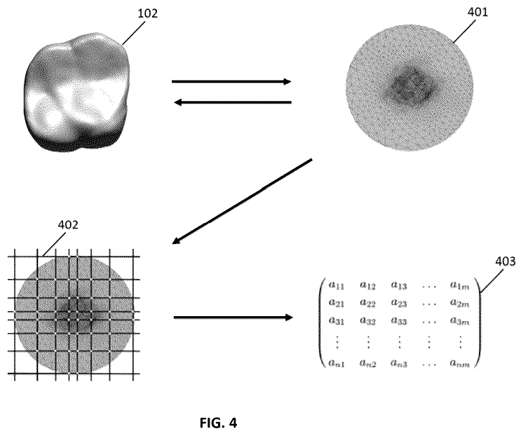

The trained neural network 300 may be suitable for processing two-dimensional input. For that purpose the input 304 may be transformed, from the 3D format such as a surface mesh, into 2D format such as a flattened mesh. This process is known as mash flattening and is further illustrated in FIG. 4. Alternatively, the 3D format of the input 304 may be transformed into 2D format by taking a plurality of virtual snapshots of the input 304. The plurality of virtual snapshots may then be utilized as the input 304.

Alternatively, the trained neural network 300 may be suitable for processing three-dimensional input such as a surface mesh, or a point cloud directly, and transforming the input 304 into 2D format may not be necessary. An example of such neural network architecture is a Point Net neural network.

Optionally, an embodiment of the method may comprise modifying and subsequently decoding the encoded tooth representation to obtain an ideal model 308 of the tooth 102. As a result of decoding, a changed geometry of the tooth 102 may be obtained. The changed geometry of the tooth 102 may show how the tooth 102 would look like in absence of any tooth wear or with tooth wear amount that would occur naturally. In this way, the ideal model 308 of the tooth 102 may be predicted.

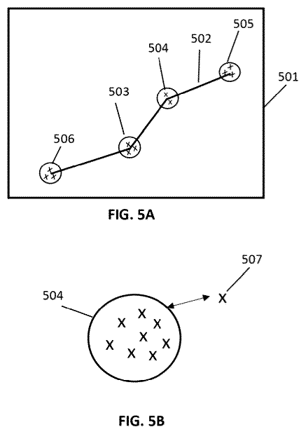

For example, it may be possible to change, in the latent space 302 of the trained neural network 300, from one cluster of latent space variables, for example from the cluster 503 corresponding to attrition to the cluster 506 corresponding to absence of tooth wear. In this way, a modified set of latent space variables may be obtained. This process of moving in the latent space 302 may be referred to as using an “tooth wear type slider” feature, which means shifting through a piece-wise linear path of the latent space 302 from one cluster to another. This is illustrated further in FIG. 5.

The function of the decoder 303 in FIG. 3B may be to convert a set of latent space variables representing the tooth 102 into an output 307. The output 307 may be of same data type as the input 304. The output 307 may be in 3D format or may be in 2D format and subsequently converted into the 3D format. In an embodiment, the decoder 303 may be a neural network, including but not limited to convolutional neural networks, dense neural networks.

In case of FIG. 3B, the output 307 represents geometry of the ideal model 308 of the tooth 102 and may be obtained by decoding the set of modified latent space variables.

In an embodiment, the method according to disclosure may comprise comparing the geometry of the tooth 102 and the geometry of the ideal model 308 of the tooth 102. The comparing may be performed by first aligning the two geometries in the 3D space and subsequently determining differences in the two geometries. Determined differences may quantify the tooth wear on the tooth 102. The differences may refer to distances between corresponding vertices of the tooth 102 and the ideal model 308 of the tooth 102. If a difference is larger than a distance threshold, tooth wear may be associated with the corresponding vertex of the tooth 102. The distance threshold may be a value of 0.3 millimeters, for example.

Determined differences that indicate tooth wear presence may be visualized as a heat map overlaying the surface of the tooth 102. Depending on the location of the determined tooth wear presence, the corresponding tooth wear type may be assigned to the tooth 102. For example attrition may be assigned to the tooth 102 if the location of the determined tooth wear presence is on occlusal surface of the tooth 102. If the location is a palatal or a facial surface of the tooth 102, further examination may be required to determine the tooth wear type.

FIG. 4 illustrates steps of transforming the tooth 102 from the 3D representation into 2D format. After the transformation, the tooth 102 in 2D format may be used as the input 304 to the trained neural network 300.

The tooth 102 may be flattened to a planar mesh 401, through a mesh flattening procedure. To flatten the tooth 102 to a planar mesh 401, a boundary may be set for cutting the tooth 102. In a three-dimensional mesh, such as 3D format of the tooth 102, places to cut include but are not limited to: anatomical features such as a gingival margin, a long axis of a tooth; geometric features such as a plane along a hemisphere; and/or any axes orthogonal to these.

Cutting the tooth 102 may result in multiple planar meshes. This may be useful for capturing more details of the tooth 102.

The boundary of the surface of the tooth 102 may then be fixed to a boundary of a planar object. The planar object may be of different shapes, including but not limited to: triangles, quadrilaterals, circles, curved quadrilaterals including circles, shapes based on the three-dimensional objects themselves.

Once the boundaries are fixed, the remaining vertices and edges are mapped to the planar object, flattening the tooth 102 to a planar mesh 401. Various embodiments may use different methods for this mapping. Initially, a matrix of each vertex′ connectivity to other vertices may be created, and solving the system of the matrices maps the coordinates of each vertex to the plane (Tutte, William Thomas. “How to draw a graph.” Proceedings of the London Mathematical Society 3.1 (1963):743-767). This connectivity may be weighted in different ways, resulting in different mappings. In various embodiments, these methods include but are not limited to: uniform weights (Tutte 1963), weights based on angles (Floater, Michael S., and Ming-Jun Lai. “Polygonal spline spaces and the numerical solution of the Poisson equation.” SIAM Journal on Numerical Analysis 54.2 (2016):797-824), weights based on cotangents (Meyer, Mark, et al. “Discrete differential-geometry operators for triangulated 2-manifolds.” Visualization and mathematics III. Springer, Berlin, Heidelberg, 2003. 35-57).

The tooth 102 in its initial 3D form and in the planar mesh 401 form may be bijective, which means it is possible for each vertex to be mapped back and forth between the tooth 102 and the planar mesh 401.

Sampled planar mesh 402 may be obtained in which each sample may be a point on the planar mesh 401. Sampling may be performed arbitrarily or based on a geometric pattern. The geometric pattern may be regular or irregular. The sampled matrix 402 in FIG. 4 illustrates an irregular geometric pattern in form of a grid used for sampling, where more information is gathered from the center of the planar mesh 401, with more relevant data, than from the edges with less relevant data. This may be an example of sampling a molar tooth, where the molar has more relevant data at the center compared to the edges. Individual samples may be taken from the intersections of the grid lines and the planar mesh 402.

Based on the individual samples, a sampled matrix 403 may be formed. The sampled matrix 403 may then be used as the input 304 to the trained neural network 300. Matrices may be particularly suitable as input form for the trained neural network 300 as many machine learning methods operate based on matrix operations.

The output 305 of the decoder 303 (FIG. 3B) may be in matrix form, from which a 3D surface of the ideal model of the tooth 102 may be reconstructed.

FIG. 5A illustrates an n-dimensional latent space 302 of the trained neural network 300, visualized as 2D space 501. The latent space 302 of the trained neural network 300 may be continuous and may comprise the plurality of clusters 503-506 of the encoded reference teeth representations. The encoded reference teeth representations may be illustrated as data points “x” within the clusters 503-506. Different clusters 503-506 may be connected by a piece-wise linear path 502 between the clusters 503-506. The clusters 503-506 may be formed, for example, by analyzing dependence of the encoded reference teeth representations to values of tooth wear type labels of the reference teeth cases.

The correlations between the encoded reference teeth representations, thereby the clusters 503-506, may be obtained by performing a statistical analysis on these variables. This may be done, in an example by using Principal Component Analysis (PCA).

Once encoding the tooth 102 into the latent space 302 of the trained neural network 300 is performed, the encoded tooth representation of the tooth 102 may be obtained. This encoded tooth representation may comprise the latent space variables representing the tooth 102 and may be illustrated with a data point 507 in FIG. 5B.

It may be determined, for example using nearest neighbor search algorithm, that the latent space variables representing the tooth 102 belong to the cluster 504, corresponding to attrition type of tooth wear.

In the example of FIG. 5B. it can be seen that the data point 507 lies outside the cluster 504. However, it may be concluded that the latent space variables representing the tooth 102 are comprised in cluster 504 because the data point 507 of interest is closest to the cluster 504. Thus, determining the cluster comprising the encoded tooth representation may comprise determining the closest cluster to the encoded tooth representation.

Optionally, the set of modified latent space variables may be obtained by shifting, from the cluster 504, to the cluster 506, where the cluster 506 is characterized, for example, by tooth wear absence. In this case it may be desired to move to this specific cluster 506 if the ideal model 308 of the tooth 102 is to be estimated. Moving within the latent space 302 between the clusters 503-506, by using piece-wise linear path between the clusters 503-506 may be achieved by utilizing continuity property of the latent space 302 to mathematically describe the piece-wise linear paths between the clusters. Other possibilities also exist, as it may be possible to move within the latent space to any desirable cluster, for example representing different age of teeth, if reference teeth have a known age parameter.

Decoding of the modified latent space variables may then be performed to obtain the output 307 of the trained neural network 300. This output 307 may be a surface geometry of the ideal model 308 of the tooth 102.

Once the ideal model 308 of the tooth 102 is obtained, it can be compared to the tooth 102. In this way a geometric difference between the tooth 102 and the ideal model 308 of the tooth 102 can be detected. The detected geometric difference may accurately quantify the tooth wear amount affecting the tooth 102. Determining the geometric difference may comprise determining distances between corresponding vertices of the tooth 102 and the ideal model 308 of the tooth 102.

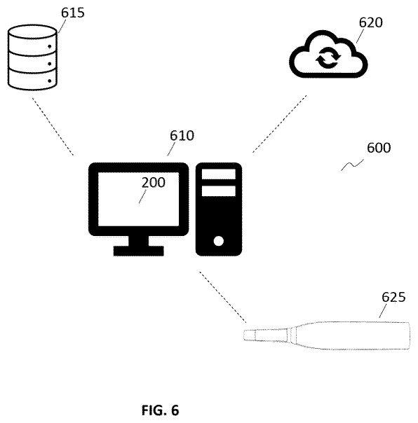

FIG. 6 illustrates a dental scanning system 600 which may comprise a computer 610 capable of carrying out the method according to any one or more embodiments of invention. The computer may comprise a wired or a wireless interface to a server 615, a cloud server 620 and an intraoral scanner 625. The intraoral scanner 625 may be capable of recording scan data of the patient's dentition.

The dental scanning system 600 may comprise a processor configured to carry out the method according to one or more embodiments of the disclosure. The processor may be a part of the computer 610, the server 615 or the cloud server 620.

A non-transitory computer-readable storage medium may be comprised in the dental scanning system 600. The non-transitory computer-readable medium can carry instructions which, when executed by a computer, cause the computer to carry out the method according to one or more embodiments of the disclosure.

A computer program product may be embodied in the non-transitory computer-readable storage medium. The computer program product may comprise instructions which, when executed by the computer, cause the computer to perform the method according to any one or more of the embodiments presented herein.

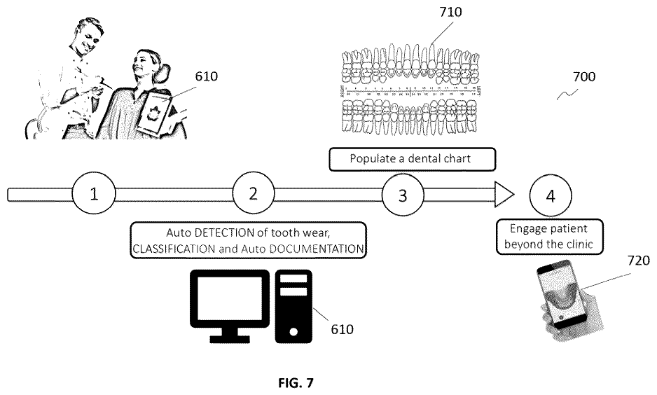

FIG. 7 illustrates an exemplary workflow 700 of the patient's visit to the dental practitioner. In step 1 the patient's oral cavity may be scanned using the intraoral scanner. In step 1 of the workflow 700 in FIG. 7, while scanning, the dental practitioner may be able to see the scanning live on a display unit of the computer 610.

At step 2 of FIG. 7, the scan data may be further analyzed utilizing one or more software applications in order to detect tooth wear presence in the patient's oral cavity. The step 2 of the workflow 700 may utilize software applications (i.e. application modules) that are configured to detect, classify, monitor, predict, prevent, visualize and/or record tooth wear that may be present in the patient oral cavity.

Step 3 of the workflow 700 exemplifies populating a dental chart 710 with information obtained in the step 2, such as for example tooth wear presence in the patient's dental cavity. The dental chart 710 may be comprised as a part of a wider patient management system.

Furthermore, as illustrated in step 4 of FIG. 7, it may be possible to connect at least a part of the dental scanning system to a smart phone 720 and thereby enable transferring of relevant information to the patient. The relevant information may comprise information on tooth wear presence in the patient's oral cavity. In this way, engagement with the patient or any other entity using the analyzed scan data beyond the dental clinic may be enabled.

Although some embodiments have been described and shown in detail, the disclosure is not restricted to such details, but may also be embodied in other ways within the scope of the subject-matter defined in the following claims. In particular, it is to be understood that other embodiments may be utilized, and structural and functional modifications may be made without departing from the scope of the present invention.

1-25. (canceled)

26. A computer-implemented method for detecting tooth wear on a virtual 3D model of a dental situation, the method comprising:
receiving, by a processor, the virtual 3D model of the dental situation;
obtaining a segmented 3D model by segmenting the virtual 3D model into a plurality of individual teeth and gingiva, wherein the segmented 3D model comprises a tooth;
encoding the tooth from the segmented 3D model into a latent space of a trained neural network to obtain an encoded tooth representation, wherein the latent space of the trained neural network is continuous and comprises a plurality of clusters of encoded reference teeth representations, wherein each cluster from the plurality of clusters of encoded reference teeth representations is representative of a tooth wear type;
comparing the encoded tooth representation to each cluster from the plurality of clusters of encoded reference teeth representations to determine a cluster comprising the encoded tooth representation;
assigning the tooth wear type to the tooth based on the determined cluster;
displaying the tooth with the assigned tooth wear type.

27. The method according to claim 26, wherein encoding the tooth from the segmented 3D model into the latent space of the trained neural network comprises feeding the information about geometry of the tooth into the trained neural network.

28. The method according to claim 26, further comprising encoding a plurality of reference teeth cases into the latent space of the trained neural network to obtain the encoded reference teeth representations, wherein each reference tooth case from the plurality of reference teeth cases comprises a reference tooth and an associated tooth wear type label.

29. The method according to the previous claim 28, wherein the tooth wear type label comprises a tooth wear severity level.

30. The method according to the previous claim 28, further comprising clustering the encoded reference teeth representations according to their corresponding tooth wear type labels to obtain the plurality of clusters of encoded reference teeth representations.

31. The method according to claim 26, wherein comparing the encoded tooth representation to each cluster from the plurality of clusters of encoded reference teeth representations to determine the cluster comprising the encoded tooth representation comprises performing a proximity search on the encoded tooth representation with respect to the each cluster from the plurality of clusters of encoded reference teeth representations.

32. The method according to claim 26, wherein comparing the encoded tooth representation to each cluster from the plurality of clusters of encoded reference teeth representations in the latent space of the trained neural network comprises using a nearest neighbor search algorithm.

33. The method according to claim 26, wherein the plurality of clusters of encoded reference teeth representations is obtained by performing a clustering analysis on the encoded reference teeth representations.

34. The method according to claim 26, further comprising obtaining a set of modified latent space variables corresponding to a cluster of encoded reference teeth representations having tooth wear type labels corresponding to absence of tooth wear.

35. The method according to the previous claim 34, further comprising decoding the set of modified latent space variables to obtain a geometry of an ideal model of the tooth.

36. The method according to the previous claim 35, further comprising detecting a geometric difference between the tooth and the ideal model of the tooth.

37. The method according to the previous claim 36, wherein detecting the geometric difference comprises determining distances between corresponding vertices of the tooth and the ideal model of the tooth.

38. The method according to claim 26, further comprising defining a path between the plurality of clusters of encoded reference teeth representations, wherein the path is piece-wise linear.

39. The method according to the previous claim 38, further comprising moving in the latent space, using the path, from a data point representing the cluster comprising the encoded tooth representation to a new data point representing the cluster of encoded reference teeth representations with tooth wear type labels corresponding to absence of tooth wear.

40. The method according to the previous claim 38, further comprising moving in the latent space, using the path, from one data point to another data point, wherein each data point is associated with a different tooth wear type for the tooth.

41. The method according to claim 26, further comprising determining a statistical parameter for each cluster of the plurality of clusters of encoded reference teeth representations as a representative of that cluster.

42. The method according to the previous claim 41, wherein comparing the encoded tooth representation to each cluster from the plurality of clusters of encoded reference teeth representations comprises performing the proximity search on the encoded tooth representation with respect to the each cluster from the plurality of clusters of encoded reference teeth representations with the statistical parameter as a target.

43. The method according to claim 26, further comprising generating automatically a treatment option for the tooth based on the determined cluster comprising the encoded tooth representation.

44. A non-transitory computer readable medium comprising instructions which, when executed by a computer, cause the computer to:
receive the virtual 3D model of the dental situation;
obtain a segmented 3D model by segmenting the virtual 3D model into a plurality of individual teeth and gingiva, wherein the segmented 3D model comprises a tooth;
encode the tooth from the segmented 3D model into a latent space of a trained neural network to obtain an encoded tooth representation, wherein the latent space of the trained neural network is continuous and comprises a plurality of clusters of encoded reference teeth representations, wherein each cluster from the plurality of clusters of encoded reference teeth representations is representative of a tooth wear type;
compare the encoded tooth representation to each cluster from the plurality of clusters of encoded reference teeth representations to determine a cluster comprising the encoded tooth representation;
assign the tooth wear type to the tooth based on the determined cluster;
display the tooth with the assigned tooth wear type.

45. A computer program product comprising instructions which, when the program is executed by a computer, cause the computer to:
receive the virtual 3D model of the dental situation;
obtain a segmented 3D model by segmenting the virtual 3D model into a plurality of individual teeth and gingiva, wherein the segmented 3D model comprises a tooth;
encode the tooth from the segmented 3D model into a latent space of a trained neural network to obtain an encoded tooth representation, wherein the latent space of the trained neural network is continuous and comprises a plurality of clusters of encoded reference teeth representations, wherein each cluster from the plurality of clusters of encoded reference teeth representations is representative of a tooth wear type;
compare the encoded tooth representation to each cluster from the plurality of clusters of encoded reference teeth representations to determine a cluster comprising the encoded tooth representation;
assign the tooth wear type to the tooth based on the determined cluster;
display the tooth with the assigned tooth wear type.
